(19) United States
(12) Patent Application Publication
(10) Pub. No.: US 2025/0367446 A1
(43) Pub. Date: Dec. 4, 2025

(54) CLOSED-LOOP NEUROMODULATION TO TREAT A CONDITION OF A BRAIN USING AN ADAPTIVE BRAIN STATE MODEL

(71) Applicant: Vanderbilt University, Nashville, TN (US)

(72) Inventors: Graham W. Johnson, Nashville, TN (US); Dario J. Englot, Nashville, TN (US)

(21) Appl. No.: 18/676,682

(22) Filed: May 29, 2024

(51) Int. Cl. A61N 1/36

(52) U.S. Cl. CPC A61N 1/36064; A61N 1/36139

(57) ABSTRACT

A condition of a brain can be treated with closed-loop neuromodulation. At least one recording electrode can record conduction data from at least a portion of the brain. At least one stimulating electrode can apply an electrical signal to another portion of the brain. A controller can execute stored instructions and a stored patient-specific model to: receive the conduction data at a time; project the conduction data through the trained patient-specific model to determine a brain state at the time; update at least one parameter of the electrical signal based on a propensity of the brain state at the time to cause an effect of the conduction of the brain; and update the trained patient specific model to include the brain state at the time and an effect of the updated parameter of the electrical signal on the brain state at the time.

### TECHNICAL FIELD

The present disclosure relates generally to treating a condition of a brain and, more specifically, to systems and methods that neuromodulate the brain (e.g., in a closed-loop) to treat the condition of the brain by implementing and continuously adapting a trained patient-specific model of brain states in response to brain conduction data.

### BACKGROUND

Epilepsy is a chronic brain condition that affects around 50 million people worldwide and causes recurring seizures. Drug-resistant epilepsy (DRE) is a subset of epilepsy where patients do not successfully respond to pharmaceutical therapy and instead rely on other techniques, such as neuromodulation therapies, to treat the DRE. Neuromodulation therapies have advanced considerably over the last fifteen years, such that now the brain can be monitored for one or more biomarker(s) of active disease states (that are constant) and at least a portion of the brain can be stimulated when at least one of the biomarker(s) is detected. Now one treatment for DRE is seizure forecasting with responsive neurostimulation (RNS). Seizure forecasting monitors for a pre-ictal signature(s), which may include a plurality of constant biomarkers, that appears immediately preceding a seizure (a short time scale biomarker) and RNS then delivers a high-energy stimulation upon detection of the pre-ictal signature(s) in an attempt to halt seizure progression. Evidence has emerged, however, that the short timescale biomarker (e.g., the pre-ictal signature(s)) and the responsive high energy stimulation may not be the most effective means to halt and/or prevent seizures.

### SUMMARY

Described herein are systems and methods that apply neuromodulation (e.g., in a closed-loop) to treat a condition of the brain by implementing and adapting a trained patient-specific model of brain states to adaptively determine a brain state at a time and determine if that brain state should trigger treatment and/or a change to a current treatment. The adaptability of the trained patient-specific model is advantageous over traditional, stagnant biomarkers at least because the trained patient-specific model can be continuously and automatically updated.

In an aspect, the present disclosure can include a system that can be used for closed-loop neuromodulation to treat a condition of the brain. The system can include at least one recording electrode configured to record conduction data from at least a portion of the brain. The system can also include at least one stimulating electrode configured to apply an electrical signal, generated and configured by a generator, to at least another portion of the brain. The electrical signal comprises at least one parameter. The system can also include a controller in electrical communication with the at least one recording electrode and the generator. The controller comprises a non-transitory memory configured to store instructions and a trained patient-specific model and a processor configured to execute the instructions and the trained patient-specific model to: receive the conduction data at a time; project the conduction data through the trained patient-specific model to determine a brain state at the time; update the at least one parameter of the electrical signal based on a propensity of the brain state at the time to cause an effect of the condition of the brain; and update the trained patient-specific model to include the brain state at the time and an effect of the updated at least one parameter of the electrical signal on the brain state at the time.

In another aspect, the present disclosure can include a method for closed-loop neuromodulation to treat a condition of the brain. The method can include: receiving, by a system comprising a processor, conduction data at a time from at least one recording electrode in communication with the processor, wherein the at least one recording electrode records conduction data from at least a portion of the brain; projecting, by the system, the conduction data at the time through a trained patient-specific model to determine a brain state at the time; updating, by the system, at least one parameter of an electrical signal based on a propensity of the brain state at the time to cause an effect of the condition of the brain, wherein the processor is further in communication with at least a generator that generates the electrical signal and provides the electrical signal to at least one stimulation electrode that applies the electrical signal to at least another portion of the brain; and updating, by the system, the trained patient-specific model to include the brain state at the time and an effect of the application of the updated the at least one parameter of the electrical signal on the brain state at the time.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a block diagram of a system for closed-loop neuromodulation to treat a condition of a brain;

FIG. 2 is an example of the controller of FIG. 1;

FIG. 3 is an example of training a patient-specific model of FIG. 2;

FIG. 4 is an example of executing the trained patient-specific model of FIG. 2;

FIG. 5 is a process flow diagram of a method for treating a condition of the brain using closed-loop neuromodulation;

FIG. 6 shows an example of synergistic adaptable closed-loop neuromodels to elucidate brain-state and deliver brain-state modulating low-energy stimulation;

FIG. 7 shows an example of a brain-state modeling process;

FIG. 8 shows an example of a custom asymmetric recurrent beta-variational autoencoder architecture;

FIG. 9 shows an example time-shift invariant loss function for multi-channel timeseries forecasting;

FIG. 10 shows an example Kullback-Leibler (KL) and Learning Rate (LR) annealing schedules;

FIG. 11 shows an example latent dimensionality reduction and spatial clustering;

FIG. 12 shows example latent spaces depicting clustered functional brain-states;

FIG. 13 shows experimental results of significant perturbation by brain-states by single-pulse electrical stimulation;

FIGS. 14-16 show training and validation of latent spaces for different subjects; and

FIG. 17 shows an example of Subject 2's seizure propensity timeline with vertical lines indicating an ictal event.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an”, and “the” can also include the plural forms unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

As used herein, the term “condition of a patient's brain” can refer to any neurological pathology, including injury, illness, or disorder, that affects the brain. Specific, but not limiting, examples of conditions of the brain can include drug-resistant epilepsy (DRE), obsessive compulsive disorder, Tourette's syndrome, major depressive disorder, schizophrenia, bipolar disorder, binge eating disorder, substance abuse disorder, or the like.

As used herein, the term “epilepsy” can refer to a neurological pathology associated with abnormal electrical activity in the brain in which a patient has two or more unprovoked seizures that occur more than 24 hours apart.

As used herein, the term “seizure” can refer to a sudden, uncontrolled burst of electrical activity in the brain. A seizure can cause changes in behavior, movements, feeling, levels of consciousness, or the like.

As used herein, the term “drug resistant epilepsy”, or “DRE”, can refer to a type of epilepsy where seizures do not successfully respond to medication therapy (e.g., at least two antiseizure medications). DRE may also be referred to as intractable, medically refractory, pharmacoresistant, or the like.

As used herein, the term “neuromodulation” can refer to the alteration of neural activity through targeted delivery of a stimulus, such as one or more of electrical stimulation, application of chemical agents, or the like.

As used herein, the term “low energy” can refer to a characterization of electrical stimulation treatment having current levels of 1 milliamps-5 milliamps and a continuous stimulation frequency of 10 Hz or less or an intermittent stimulation with frequency of greater than 10 Hz for less than 1 second followed by at least 1 second of no stimulation.

As used herein, the term “electrode” can refer to a solid electrical conductor that carries electric current into one or more non-metallic elements (e.g., within a patient's body). Electrodes can record data and/or deliver electrical stimulation and can be internal electrodes (e.g., intracranial electrodes) and/or surface electrodes.

As used herein, the term “conduction data” can refer to electrical activity (e.g., one or more signals) recorded from a patient's brain by one or more electrodes. For example, the conduction data can be recorded using electroencephalography (also referred to as EEG), a test that measures electrical activity a patient's brain. Electrodes used for EEG can be external, internal, or the like. One example of EEG is intracranial EEG (also referred to as iEEG) where EEG is obtained with intracranial electrodes, an example of which is stereoelectroencephalography (also referred to as SEEG). Another example of EEG is electrocorticography (ECOG), where EEG is acquired by strips or grids of electrodes implanted over the bare cortex in subdural space. A further example of EEG uses electrodes attached to the scalp.

As used herein, the term “latent space”, also referred to as “latent feature space” and “embedding space”, can refer to a multi-dimensional space that encodes a meaningful representation of characteristics of a set of data (e.g., embedded within a manifold in which items resembling each other are positioned closer to one another). The latent space can be high-dimensional, complex, and non-linear. The latent space provides a compressed understanding to a computer through a spatial representation.

As used herein, the term “brain state”, also referred to as “brain-state”, can refer to a representation of electrical activity in one or more areas of the brain at a given time.

As used herein, the term “patient”, also referred to as subject and other similar terms, can refer to any warm-blooded organism, including, but not limited to, a human being, a pig, a rat, a mouse, a dog, a cat, a goat, a sheep, a horse, a monkey, an ape, a rabbit, a cow, etc.

### II. Overview

Neuromodulation for drug resistant epilepsy (DRE) (and other neurological pathologies) has advanced considerably in recent years and has made closed-loop neuromodulation possible. The brain can be monitored for potential biomarkers of active disease states and stimulation can be applied based on a feedback loop for the biomarkers. In fact, Responsive Neurostimulation (RNS) is used for patients with DRE to detect immediate pre-ictal signatures and deliver high-energy stimulation in an attempt to abort seizure progression. However, recent research has shown that RNS based on pre-ictal signatures may be too late to effectively halt seizures and that low-energy stimulation may be more effective (this is in alignment with clinical observations that seizures tend to occur on long-range periodic timescales).

As such, described herein are systems and methods that can neuromodulate a patient's brain to treat a condition of the brain (including DRE) based on an adaptable trained patient-specific model of brain states significantly before current pre-ictal signatures (e.g., on a long timescale). Indeed, the trained patient-specific model can be continuously and automatically updated as conduction data of the patient is recorded to further personalize and improve the treatment. Currently monitored biomarkers struggle to accurately forecast seizures in many instances, which may be at least partially due to their being unidimensional, as well as having too short a time scale. The trained patient-specific model can be multi-dimensional, accounting for many dimensions of data in determining and identifying the vast quantity of possible brain states and their long-term consequences on a given patient. The systems and methods described herein utilize novel signal processing and normalization, a custom asymmetric variational autoencoder, and a novel loss paradigm to elucidate a trained patient-specific model of brain-state(s) that can further be trained to deliver proper neurostimulation (e.g., low energy) over long time periods to maintain satisfactory brain states of a patient. Brain state at a given time can be quantified on a gradient scale from low propensity to high propensity (e.g., for a seizure or other effect of a condition of the brain) in a method similar to a forecasting a tornado (e.g., no alert, watch, warning). It should be noted that the trained patient-specific model can be nearly infinitely multi-dimensional (e.g., 512 dimensions or more) well beyond the limits of human comprehension and unaided computations.

### III. Systems

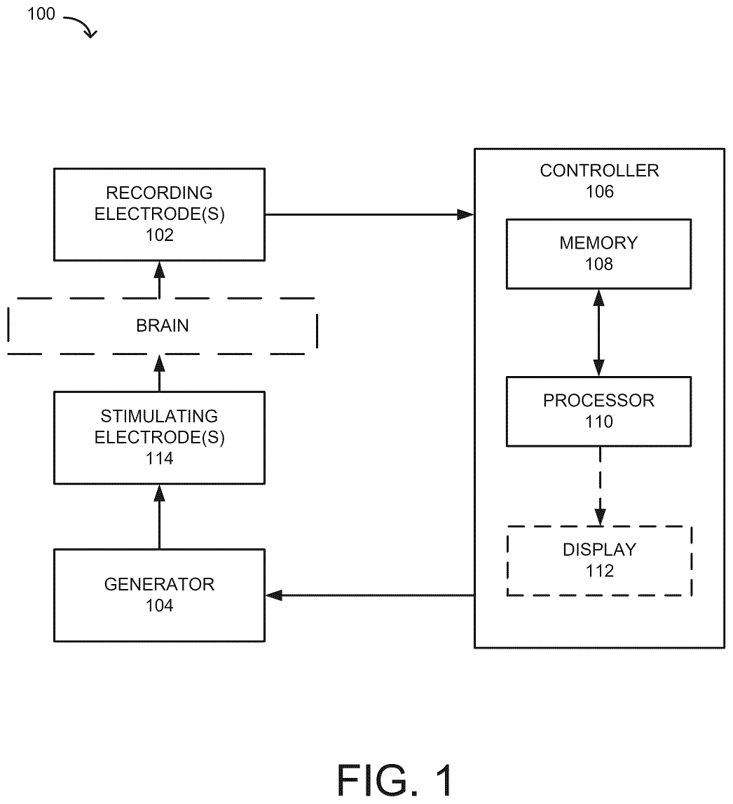

An aspect of the present disclosure can include a system 100 (FIG. 1) that can treat a condition of a patient's brain using neuromodulation (e.g., closed-loop neuromodulation). The condition of the patient's brain can refer to any neurological pathology, including injury, illness, or disorder, that affects the brain. Specific examples of conditions of the brain can include one or more of epilepsy, including drug-resistant epilepsy (DRE), obsessive compulsive disorder, Tourette's syndrome, major depressive disorder, schizophrenia, bipolar disorder, binge eating disorder, substance abuse disorder, or the like. For instance, neuromodulation with the system 100 can prevent and/or control seizures, obsessions and/or compulsions, tics, depressive episodes, schizophrenic symptoms, bipolar swings, binging or substance compulsions, or the like. Alternatively, the system 100 can be used in applications that use conduction in the brain to perform a function, such as BCI (brain-computer interfaces). Generally, neuromodulation is the alteration of neural activity through targeted delivery of a stimulus (e.g., electrical, chemical, magnetic, radiation (e.g., heat, light, or the like), etc.). The neuromodulation, in some instances, can be electrical neuromodulation that can stimulate one or more portions of the brain with a low energy electrical signal over long time periods. It should be understood that while only electrical neuromodulation is described in detail, magnetic stimulation (as well as other therapies traditionally used to treat neurological conditions) is also contemplated as able to work in a similar manner, and only electrical modulation is referred to herein solely for ease of description.

As shown in FIG. 1, the system 100 can include one or more recording electrodes (recording electrode(s) 102) that can record conduction information from at least a portion of the patient's brain (shown in FIG. 1 as the dashed box labeled brain). Conduction data can refer to electrical activity recorded from the patient's brain. For example, the conduction data can be recorded using electroencephalography (also referred to as EEG), a test that measures electrical activity a patient's brain. Recording electrode(s) 102 can be at least one of external, internal, intracranial, or the like. For instance, the recording electrode(s) 102 can include one or more intracranial electrodes for intracranial EEG (also referred to as iEEG) or stereoelectroencephalography (also referred to as SEEG). In another instance, the recording electrode(s) 102 can be one or more electrodes implanted over the cortex in subdural space for electrocorticography (ECOG). In a further instance the recording electrode(s) 102 can be scalp electrodes configured for scalp EEG recording. In each case, the conduction data can provide a representation of electrical activity in one or more areas of the brain at a given time. The recording electrode(s) 102 can be any number N that is one or greater.

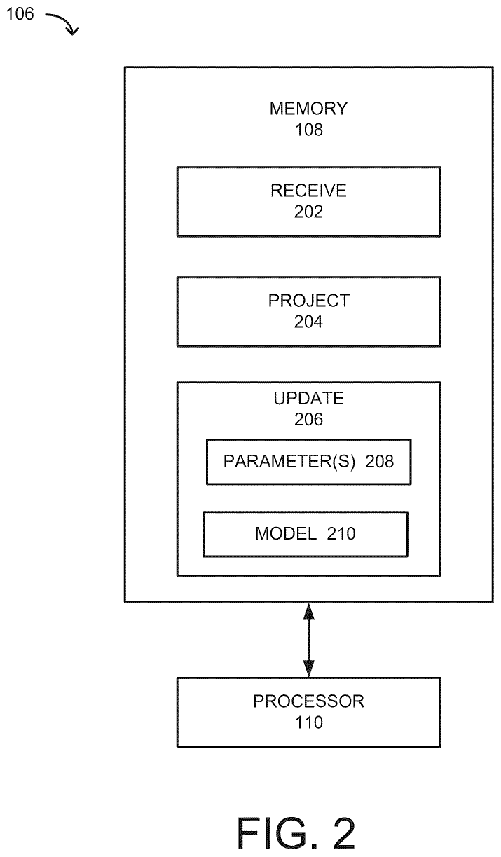

Conduction data recorded by the recording electrode(s) 102 (N channels of data corresponding to the N recording electrodes 102) can be sent to controller 106. The conduction data can be recorded and transmitted at one or more frequencies. The recording electrode(s) 102 and the controller 106 can be in wired and/or wireless electrical communication. The controller 106 can include memory 108 (which is non-transitory) and a processor 110 (e.g., a microprocessor, a computing device, a state machine, a signal processing chip, or the like). In some instances, at least a portion of the memory 108 and processor 110 can be embedded within the same device (e.g., a microprocessor). In other instances, the memory 108 and the processor 110 can be entirely separate devices. The memory 108 can store instructions related to training and execution of a trained patient-specific model and the trained patient-specific model itself. The processor 110 can execute the instructions and the trained patient-specific model, for instance, to determine brain states from the conduction data and whether, and which one or more parameters of the electrical neuromodulation, to update the neuromodulation treating the condition of the brain (more detail shown in FIG. 2). The controller 106 may also, in some instances, have a display 112 (or other type of output device for visual, audible, or tactile output) and/or an input device (not shown, for inputting one or more instructions, limits, additional data, or the like).

Upon determining that at least a portion of the conduction data at a time corresponds to a brain state indicating a need for neuromodulation and/or a change in neuromodulation (e.g., a high propensity brain state, described in further detail below), the controller 106 can determine (if no neuromodulation is being applied) or update (if neuromodulation is already being applied) one or more parameters of an electrical signal (e.g., corresponding to shape, current, voltage, amplitude, frequency, pulsation, timing, etc.) and send the one or more new or updated parameters to a generator 104. The generator 104 may be a standalone device in electrical communication (wired and/or wireless) with the controller 106 and the stimulating electrode(s) 114 (as shown), part of the controller 106, part of a system including the stimulating electrode(s) 114, etc.). The generator 104 can receive the one or more new or updated parameters and create or update the electrical signal based on the new or updated parameters. The electrical signal can be sent to one or more stimulating electrodes (stimulating electrode(s) 114) for application of the configured electrical signal to at least another portion of the brain for neuromodulation. The at least the other portion of the brain can include at least a portion of the same portion of the brain monitored by the recording electrode(s) 102, different from the portion of the brain monitored by the recording electrode(s), and/or at least partially the same and at least partially different. The stimulating electrode(s) 114 can be internal electrodes (e.g., intracranial electrodes) and/or surface electrodes (e.g., positioned on the scalp). In some instances, the recording electrode(s) 102 and the stimulating electrode(s) 114 can be the same electrodes. In other instances, the recording electrode(s) 102 and the stimulating electrode(s) 114 can be unique and distinct from one another.

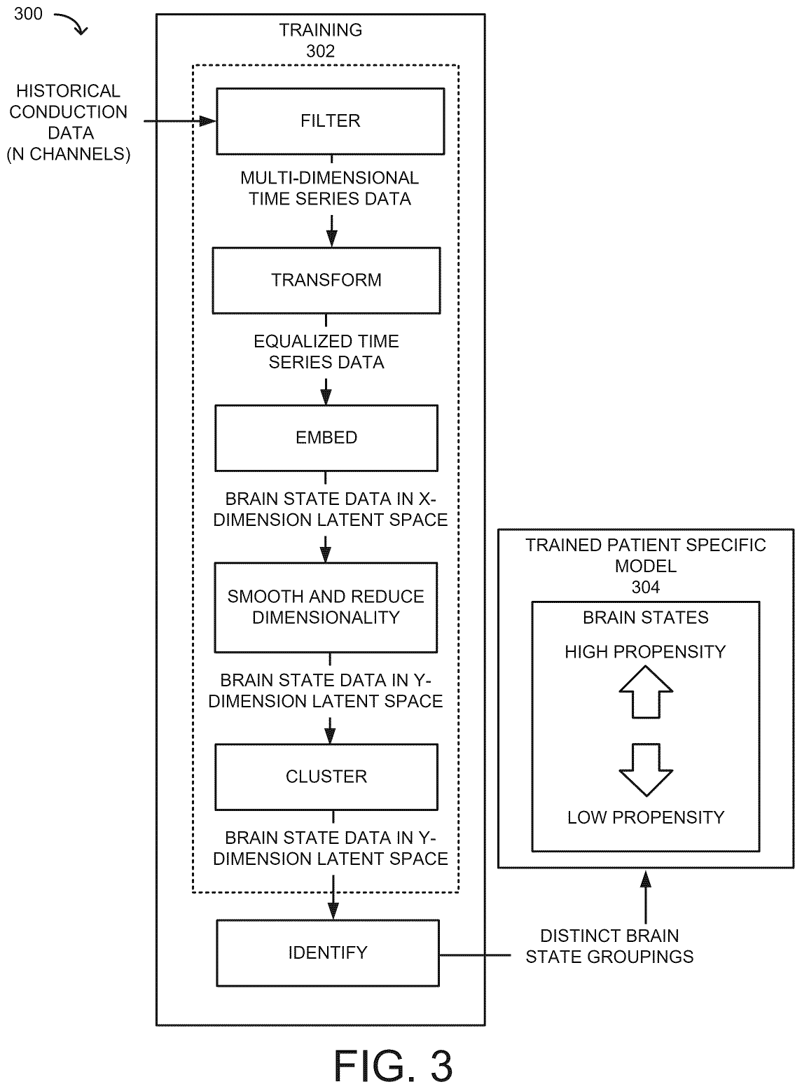

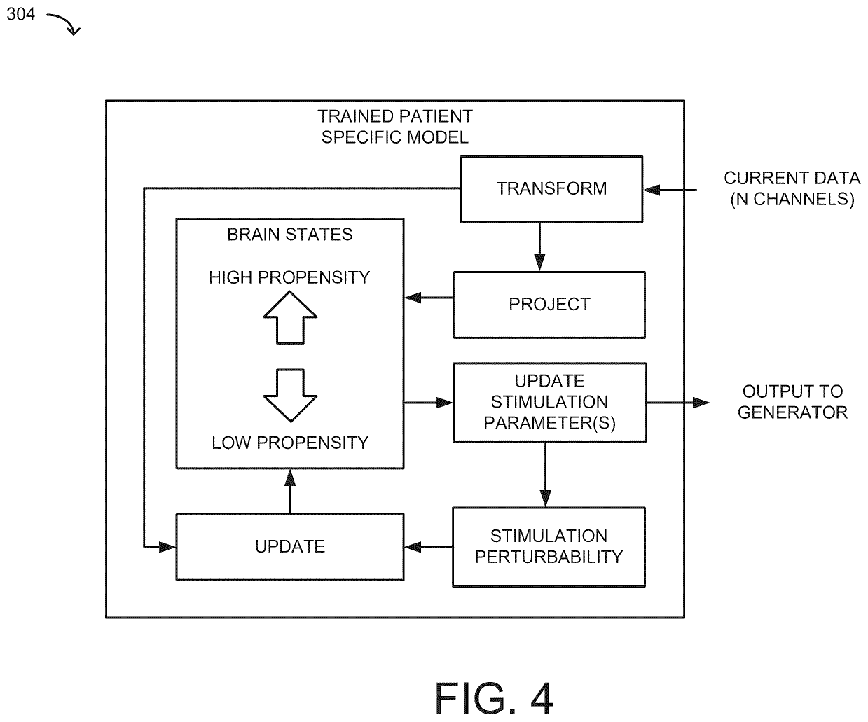

The system 100 can be a closed-loop between the recording electrode(s) 102, the controller 106, and the generator 104/stimulating electrode(s) 114. To implement the neuromodulation in the closed-loop, the controller 106 can train, implement, and adapt the trained patient-specific model of brain states to adaptively determine a propensity of a brain state at a given time towards one or more effects or symptoms of a condition of the brain of the patient and determine whether that brain state should trigger treatment and/or a change to a current treatment. An example of the functionality of the controller 106 is shown in FIG. 2, further details are discussed with respect to FIGS. 3 and 4. Conduction data recorded by the recording electrodes can be received at a time (receive 202). The conduction data can then be projected through the trained patient-specific model to determine a brain state at the time and the brain state's propensity (project 204). For example, in use after training, the processor 110 can execute the trained patient-specific model and can process the conduction data at the time into brain state data at the time to be compatible with the trained patient-specific model. The brain state data can then be projected through the trained patient-specific model to determine the propensity of the brain state at the time and whether that brain state should trigger treatment and/or a change to a current treatment.

The trained patient-specific model can employ a multi-dimensional latent space to determine whether the brain state at the time has a propensity to cause an effect on a condition of the brain. For instance, the brain state at the time can be run through the multi-dimensional latent space and analyzed with respect to a multitude of brain states with known effects on the condition of the brain to determine the current propensity to cause an effect on the condition of the brain. It should be noted that the this is far from a simple one-to-one comparison (or even a plurality of one-to-one comparisons) but instead includes multi-dimensional comparisons of positions and calculations of a plurality of interacting signs and brain states that a person cannot accomplish in the mind. As an example, the positions can correspond to cautions similar to tornado watches and warnings. In one area, the positions can correspond to no watch (low propensity), another area can correspond to a watch if some early signs appear (mid-level propensity), while another area can correspond to a warning when more or stronger signs appear (high propensity). It should be understood that these are only examples, and that the propensity can be on a gradient that can include many different propensities that may or may not be pointed in nature. The trained patient-specific model can include historical data with known outcomes for the comparison but need not include each of the specific brain states being compared during use (e.g., the patient-specific model can predict propensity even for brain state data that it has not yet seen as it is believed that no two brain states are entirely identical in latent space). It should be further noted that, while not wishing to be bound by theory, the exact time course of brain states in latent space before a given seizure cannot be reliably labeled (e.g., will or will not result in an effect) so propensity is determined rather than categorization of brain states. When the brain state indicates a propensity to cause an effect on the condition of the brain, at least one parameter of the electrical signal for the neuromodulation (parameter(s) 208) and the trained patient specific model (model 210) can be updated (updated 206). As part of the updating the at least one parameter can be updated and sent to the generator via the processor 110 and the trained patient-specific model can be updated to include that brain state, the effect on the patient, and/or the effect of the change in the one or more parameters on the patient.

Shown in FIG. 3 an example 300 of how the trained patient specific model is trained for each unique patient. Historical conduction data (from N-channels corresponding to N recording electrodes) from a given previous time period can be input into a training 302 module. The training 302 module can receive the historical conduction data and filter the historical conduction data into multi-dimensional time series data signals for the N channels. The filtering can, for instance, include a zero-phase shift 5^{th} order Butterworth filter with passbands of 1 Hz-59 Hz, 61 Hz-119 Hz, and 121 Hz-179 Hz. The historical conduction data may also be re-sampled to the lowest sample rate of all the historical conduction data. For example, if the sampling rate of the historical conduction data ranged from 512 Hz to 2048 Hz, then the historical conduction data can all be resampled at 512 Hz. The training 302 module can then equalize the multi-dimensional time series data signal using a zero-centered one-dimensional histogram equalization (ZHE) scheme to form equalized time series data. For instance, the signal can be split into the positive and negative domains, then a 10,000-bin histogram can be filled for each domain from 0 to the absolute value of highest voltage. Then the linear transfer function from the time series signal to the equalized signal can be calculated using the cumulative distribution function for the positive and negative domain separately. The result is a signal that preserves the physiological meaning of zero, but has data evenly distributed between −1 and 1. A normalization scheme can then be applied, such as normalizing all the subject's time series to the first, for instance, 24 hours, of data to produce equalized time series data for each channel.

Then, the training 302 module can embed the equalized time series data into a multi-dimensional latent space using an asymmetric recurrent variational autoencoder (AR-BVAE) to form the brain state data in X dimension-latent data space (e.g., having 512 dimensions, but may have any number of dimensions limited only by processing power of the computer and the highest sampling rate of the conduction data) and, in some instances, data forecasting, smooth the latent data (e.g., with a 10-second averaging window and 1 second stride, but any sized-averaging window and/or stride can be used limited by the processing power of the computer). It should be noted that 512 dimensions is far beyond the range of human comprehension.

For instance, the equalized time series data can be compressed into short data epochs (e.g., 0.5 seconds, but can be any short time segment) in 512-dimensional latent space. From there the immediate future (e.g., 0.125 seconds or the like) of all channel data can be simultaneously forecast. The AR-BVAE can be architected to accept N channels of X number of samples (for instance 88-186 channels by 256 samples) and can have multiple layers. The top layer can utilize a Gated Recurrent Unit (GRU) to interface with the input data (the equalized time series data in epochs for the N channels and X samples). The GRU can learn short- and long-range signal features. The GRU can be three layers and bidirectional, with a resulting hidden dimension of 2N that can be subsampled every eight brain states. While not wishing to be bound by theory, this can help the GRU to maintain learning capacity and not be forced to forget data motifs across the forward and reversed sequence lengths. The subsampled hidden states can then be flattened and fed into the B-VAE. The B-VAE can include fully-connected layer feeding into mean and log-variance layers that can be followed by a standard noise-injection reparameterization trick to determine the latent space. The latent space can be regularized by Kulback-Leibler (KL) Divergence and set to a number Y (e.g., 512). The decoder can output an N×64 sample forecast on all channels simultaneously that are compressed into 1×Y (e.g., 512) latent dimensions, which, not wishing to be bound by theory, best embed the necessary information to forecast the next N×64 timepoints in the original input signal. A dropout ratio (e.g., 0.1, 0.2, 0.3, or the like) can be used on the middle fully connected layer of the decoder (e.g., to promote concise and meaningful latent embeddings).

The decoding portion of the B-VAE can be asymmetric, to account for this a custom loss function (called Circular Minimum Hyperbolic Cosine Loss (cMin-LogCosh)) can be applied that allows for varying time shift of the forecasted signals. For instance, the custom loss function can be based on the hyperbolic cosine loss function and the N×64 predicted values can be wrapped in a circle with a stride of one sample and the LogCosh loss calculated every stride resulting in 64 individual loss values. The minimum LogCosh loss can be determined and returned for backpropagation through the model. Additional data transformation may also be used to increase stability, such as learning rate annealing. The 512 dimensional data can also be smoothed with a 10 second averaging window and a 1 second stride to greatly increased the signal-to-noise ratio prior to the manifold approximation.

The training 302 module can then reduce the dimensionality of the smooth latent data through a pairwise controlled manifold approximation and projection (PaCMAP) (e.g., from 512 to 10), cluster the PaCMAP reduced dimensional space data (10 dimensional data, still beyond the range of human comprehension). The reduced dimensional space data can then be fed into a Hierarchical Density Based Spatial Clustering of Applications of Noise (HDBSCAN) for clustering. Distinct brain state grouping(s) for the historical data can be identified in the reduced dimension and clustered data. The historical conduction data over which the trained patient-specific model is trained can be brain-state data for a previous time period (e.g., of more than 10 minutes, 30 minutes, 1 hour or more, 1 day or more, 3 days or more, etc.). The distinct brain state groupings discovered by the training 302 module can be used to form the high propensity to low propensity designations (e.g., on a gradient) in the trained patient-specific model 304 (shown in greater detail in FIG. 4). It should be understood that the training can be done before the model is used (e.g., using historical data from the patient, historical data from patients suffering from the same general condition, etc.) and can be continued for each “update” during implementation of the model for closed-loop neuromodulation.

Referring now to FIG. 4, illustrated is an example of using the trained patient specific model 304 in greater detail. The trained patient specific model 304 can be trained as shown in FIG. 3. Then new conduction data (recorded by the recording electrodes) can be input into trained patient specific model. The new conduction data can undergo the same transformation steps as described above with respect to the historical conduction data in FIG. 3 (e.g., take the conduction data and process and filter the conduction data into multi-dimensional time series data for the N channels, equalize the multi-dimensional time series date using a zero-centered one-dimensional histogram equalization (ZHE) to form equalized time series data, embed the equalized time series data into a multi-dimensional latent space using an asymmetric recurrent variational autoencoder (AR-BVAE) to form the brain state data in X dimension-latent data space (e.g., 512 dimensions) and, in some instances, data forecasting, smooth the X-dimensional latent data (e.g., with a 10-second averaging window and 1 second stride), reduce the dimensionality of the data through a pairwise controlled manifold approximation and projection (PaCMAP) (e.g., from 512 to 10), cluster the PaCMAP reduced dimensional space (10 dimensional data) with a Hierarchical Density Based Spatial Clustering of Applications of Noise (HDBSCAN); new distinct brain state grouping(s) can be identified in the reduced dimension and clustered data). The new distinct brain state groupings for the time can be projected through the trained patient-specific model and analyze with respect to previously identified groupings (from the training). A propensity can be assigned to the brain state groupings at the time and stimulation parameters can be updated based on the propensity and sent to the generator. Stimulation perturbability can be determined and the determination can be then fed back into the training 302 module with the current conduction data (that may be processed) for further updating the model (e.g., to make the model better with more current historical data).

### IV. Methods

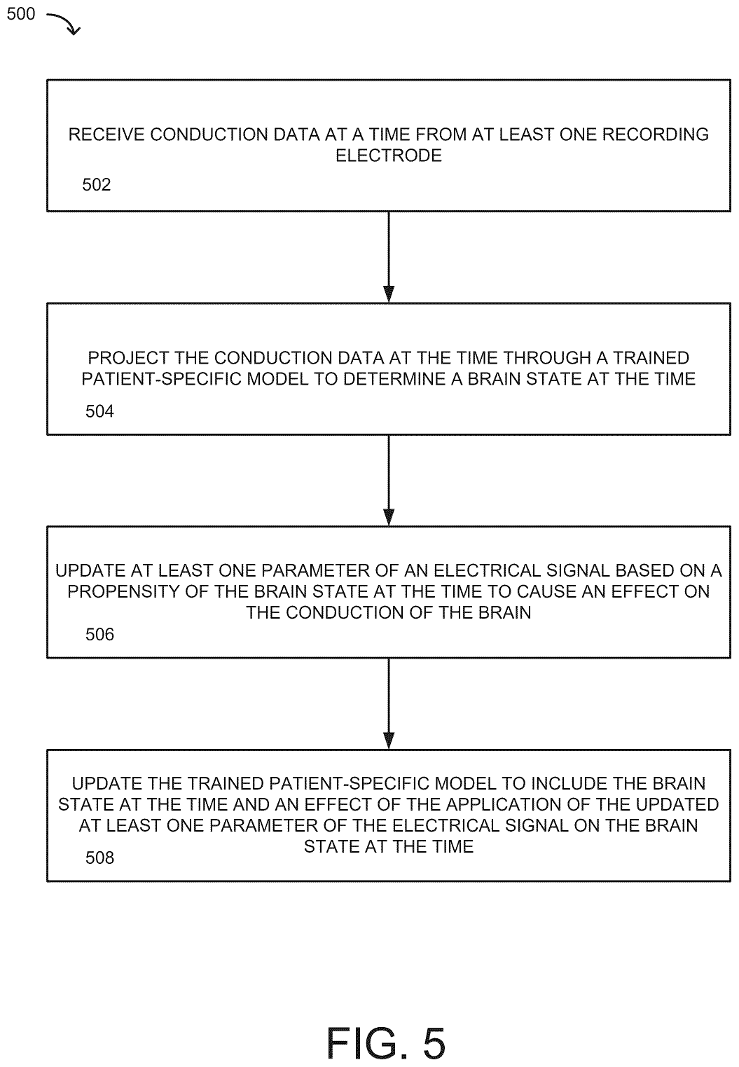

Another aspect of the present disclosure can include method 500 (FIG. 5) for treating a condition of the brain using closed-loop neuromodulation. The method 500 can be executed using the system 100 (shown in FIG. 1 and modified by FIGS. 2-4). It should be understood that system 100 includes one or more recording electrode(s) that record conduction data from an area of at least part of the brain, a controller that receives the conduction data, implements a model of brain state to process the conduction data and determine a likelihood of the brain state contributing to a brain condition, and (if necessary) output a change of one or more parameters of a stimulation, a generator that receives the output and changes the one or more parameters and generates the stimulation, and one or more stimulating electrode(s) to deliver the stimulation to another at least part of the brain (which may be the same and/or different than where the conduction data is recorded).

For purposes of simplicity, the method 500 is shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the method 500, nor is method 500 limited to the illustrated aspects.

The method 500 illustrates the actions of the controller to performed closed-loop neuromodulation to treat a condition of the brain. It will be understood that the controller can have a non-transitory memory and a processor. The non-transitory memory can store the instructions of method 500, as well as a patient-specific model (e.g., trained for a certain patient). The processor can access the memory and execute the instructions with the trained patient-specific model. Each of the steps of method 500 can be executed by the processor of the controller of system 100, for example. The method 500 can continuously modify the neuromodulation in a closed-loop to treat the condition of the patient's brain. The condition of the patient's brain can be a neurological pathology, including injury, illness, or disorder, that affects the brain. Specific examples of conditions of the brain can include one or more of epilepsy, such as drug-resistant epilepsy (DRE), obsessive compulsive disorder, Tourette's syndrome, major depressive disorder, schizophrenia, bipolar disorder, binge eating disorder, substance abuse disorder, or the like. The neuromodulation can treat and/or prevent one or more symptoms or effects of the condition of the brain, such as seizures for epilepsy. Furthermore, the neuromodulation can keep the patient in a pre-identified desirable brain state with a high latent space distance from the subclinical onset of undesirable disease symptoms.

At 502, conduction data for a time can be received from at least one recording electrode (e.g., positioned in, on, and/or above the portion of the brain of the patient). The at least one recording electrode can be any number N greater than one and can be sent over N channels to the processor. The at least one recording electrode can be in communication (wired and/or wireless) with the processor (in some instances, in communication with the non-transitory memory which is in communication with the processor).

At 504, the conduction data at the time can be projected through a trained patient-specific model (e.g., trained as shown in FIG. 3) to determine a brain state at the time. The conduction data at the time can be transformed into brain state data at the time that is compatible with the trained patient-specific model (as shown at least in FIG. 4). The transformation can include filtering the conduction data into multi-dimensional time series data signals for the N channels. The filtering can, for instance, include a zero-phase shift 5^{th} order Butterworth filter with passbands of 1 Hz-59 Hz, 61 Hz-119 Hz, and 121 Hz-179 Hz. The conduction data may also be re-sampled to the lowest sample rate of all the conduction data (e.g., if the channels have different sampling rates). For example, if the sampling rate of the conduction data ranges from 512 Hz to 2048 Hz, then the conduction data can all be resampled at 512 Hz to form a multi-dimensional time series data signal.

The trained patient-specific model can then equalize the multi-dimensional time series data signal using a zero-centered one-dimensional histogram equalization (ZHE) scheme to form equalized time series data. For instance, the signal can be split into the positive and negative domains, then a 10,000-bin histogram can be filled for each domain from 0 to the absolute value of highest voltage. Then the linear transfer function from the time series signal to the equalized signal can calculate using the cumulative distribution function for the positive and negative domain separately. The result is a signal that preserves the physiological meaning of zero, but has data evenly distributed between −1 and 1. A normalization scheme can then be applied, such as normalizing all the subject's time series to the first, for instance, 24 hours, of data to produce equalized time series data for each channel.

Then, the equalized time series data can be embedded into a multi-dimensional latent space using an asymmetric recurrent variational autoencoder (AR-BVAE) to form the brain state data in X dimension-latent data space (e.g., having 512 dimensions, but may have any number of dimensions limited only by processing power of the processor and the highest sampling rate of the conduction data) and, in some instances, data forecasting, smooth the latent data (e.g., with a 10-second averaging window and 1 second stride, but any sized-averaging window and/or stride can be used limited by the processing power of the computer). It should be noted that 512 dimensions is far beyond the range of human comprehension.

For instance, the equalized time series data can be compressed into short data epochs (e.g., 0.5 seconds, but can be any short time segment) in 512-dimensional latent space. From there the immediate future (e.g., 0.125 seconds or the like) of all channel data can be simultaneously forecast. The AR-BVAE can be architected to accept N channels of X number of samples (for instance 88-186 channels by 256 samples) and can have multiple layers. The top layer can utilize a Gated Recurrent Unit (GRU) to interface with the input data (the equalized time series data in epochs for the N channels and X samples). The GRU can learn short- and long-range signal features. The GRU can be three layers and bidirectional, with a resulting hidden dimension of 2N that can be subsampled every eight brain states. While not wishing to be bound by theory, this can help the GRU to maintain learning capacity and not be forced to forget data motifs across the forward and reversed sequence lengths. The subsampled hidden states can then be flattened and fed into the B-VAE. The B-VAE can include fully-connected layer feeding into mean and log-variance layers that can be followed by a standard noise-injection reparameterization trick to determine the latent space. The latent space can be regularized by Kulback-Leibler (KL) Divergence and set to a number Y (e.g., 512). The decoder can output an N×64 sample forecast on all channels simultaneously that are compressed into 1×Y (e.g., 512) latent dimensions, which, not wishing to be bound by theory, best embed the necessary information to forecast the next N×64 timepoints in the original input signal. A dropout ratio (e.g., 0.1, 0.2, 0.3, or the like) can be used on the middle fully connected layer of the decoder (e.g., to promote concise and meaningful latent embeddings).

The decoding portion of the B-VAE can be asymmetric, to account for this a custom loss function (called Circular Minimum Hyperbolic Cosine Loss (cMin-LogCosh)) can be applied that allows for varying time shift of the forecasted signals. For instance, the custom loss function can be based on the hyperbolic cosine loss function and the N×64 predicted values can be wrapped in a circle with a stride of one sample and the LogCosh loss calculated every stride resulting in 64 individual loss values. The minimum LogCosh loss can be determined and returned for backpropagation through the model. Additional data transformation may also be used to increase stability, such as learning rate annealing. The 51-dimensional data can also be smoothed with a 10 second averaging window and a 1 second stride to greatly increased the signal-to-noise ratio prior to the manifold approximation.

The dimensionality of the smooth latent data can then be reduced through a pairwise controlled manifold approximation and projection (PaCMAP) (e.g., from 512 to 10), cluster the PaCMAP reduced dimensional space data (e.g., 10 dimensional data, still beyond the range of human comprehension). The reduced dimensional space data can then be fed into a Hierarchical Density Based Spatial Clustering of Applications of Noise (HDBSCAN) for clustering. Distinct brain state grouping(s) for the conduction data at the time can be identified in the reduced dimension and clustered data. The distinct brain state groupings for the time can be projected through the trained patient-specific model. The projection can determine the propensity of the brain state at that time to cause an effect of the condition of the brain. The trained patient specific model does more than compare the current data to previous data, instead the trained patient specific model can analyze the current data with respect to previous data and can even assign a propensity to brain states that have not been seen before based on the analysis. It should be noted that the trained patient-specific model is trained over brain state data from a previous time period of at least one hour, but more preferably at least one day.

At 506, at least one parameter of an electrical signal can be updated based on a propensity of the brain state at the time to cause an effect on the condition of the brain. For instance, if the propensity is considered a high propensity to cause an effect on the condition of the brain (e.g., to cause a seizure or other symptom in the future) then the electrical signal can be modulated (or started if no stimulation was being applied before) to at least partially prevent the effect (or symptom) The processor is in communication with at least a generator that generates the electrical signal and provides the electrical signal to at least one stimulation electrode that applies the electrical signal to the at least another portion of the brain. At 508, the trained patient-specific model can be updated to include the brain state at the time and an effect of the application of the updated at least one parameter of the electrical signal on the brain state at the time. In such a manner the trained patient-specific model is continually updating, adapting, and improving treatment for the patient.

### V. Experimental

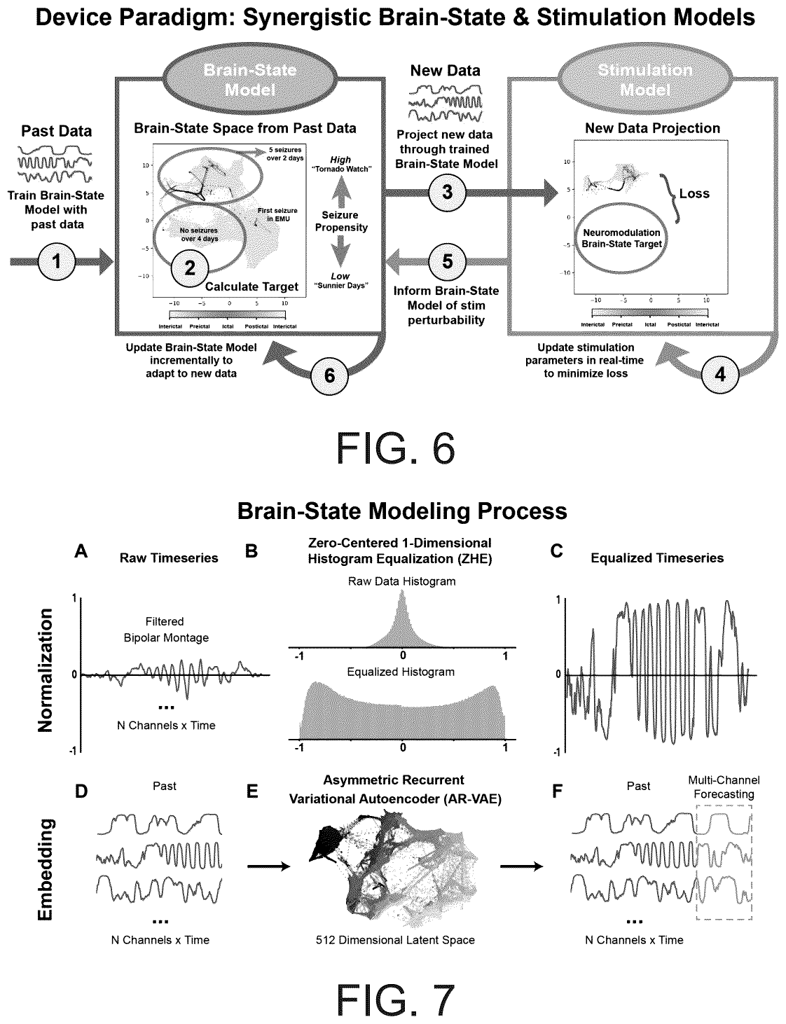

The future of closed-loop adaptive neuromodulation for drug-resistant epilepsy (DRE), as well as other neurological pathologies, relies on effectively quantifying long timescale disease propensity in a smooth and continuous distribution with a functional brain-state for effective device feedback (see example of FIG. 6). The following experiment shows the demonstration and validation of quantifying long timescale disease propensity in a smooth and continuous distribution using stereotactic-electroencephalography (SEEG, a subset of intracranial electroencephalography (iEEG)) timeseries from patients undergoing pre-surgical workup for DRE.

TABLE 1
contains information about the Epilepsy Brain-State
Postulates referenced in this Experimental section.
Epilepsy Brain-State Postulates

DEFINITIONS
Latent space: The regularized multidimensional space of possible brain-
states constructed from previously observed phenomena (e.g. intracranial
electroencephalography).
Brain-state/Embedding: The exact mapped location within the latent space.
Brain-state is synonymous with ‘embedding’ in this context.
Latent distance: The mathematical distance between two brain-states in the
high-dimensional latent space, as defined by metrics like Euclidean,
Manhattan, or angular distance. The higher the latent distance, the more
‘different’ the brain states are. Words like ‘far’ and ‘furthest’ are used to
indicate brain-states separated by a large latent distance.
POSTULATES
Postulate 1: NO TWO PRE-ICTAL BRAIN-STATES ARE IDENTICAL.
Implication: Exact latent space embedding of previous events only serves
as approximate landmark for future events. Furthermore, different types of
ictal events (e.g. subclinical, focal aware, focal impaired-aware, focal to
bilateral tonic-clonic) likely embed into different latent regions.
Postulate 2: TEMPORAL EVOLUTION FROM PRE-ICTAL TO ICTAL
BRAIN-STATE CAN VARY.
Implication: The exact time course of brain-states in latent space before a
given seizure cannot be reliably used as a label for latent space
construction or post-hoc interpretation. Specifically, not all brain-states
adjacent to a previously verified pre-ictal state will evolve into a seizure in
the same manner - there is an inherent level of stochasticity that is difficult
to model. Thus, latent space construction benefits from a purely
unsupervised/self-supervised model to avoid bias.
Postulate 3: THE BRAIN-STATE ‘FURTHEST’ FROM OBSERVED
PRE-ICTAL AND ICTAL ACTIVITY IS ILL-DEFINED.
Implication: In a patient currently experiencing multiple seizures, it is
likely that few to no ‘stable’ brain-states far from ictal activity are
observed and mapped into the latent space. Thus, a theoretical optimal
brain-state target for closed-loop neuromodulation is likely not readily
defined by previously observable brain states. Furthermore, there is likely
a constellation of potentially stable brain states far from potential ictal
activity.

### Methods

The goal of this work was to develop a generalizable framework for a functional brain-state map from high-quality intracranial electrophysiological timeseries to be used with closed-loop neuromodulation. To develop and validate the brain-state model architecture, a cohort of approximately 17,000 hours (16.3 TB of 32-bit precision) of continuous Stereoelectroencephalography (SEEG) data from 118 patients with drug resistant epilepsy (DRE) undergoing SEEG presurgical evaluation at Vanderbilt University Medical Center (VUMC) were utilized. This study was approved by Vanderbilt's Institutional Review Board and all patients provided informed consent.

### Brain-State Modeling.

### Data Acquisition.

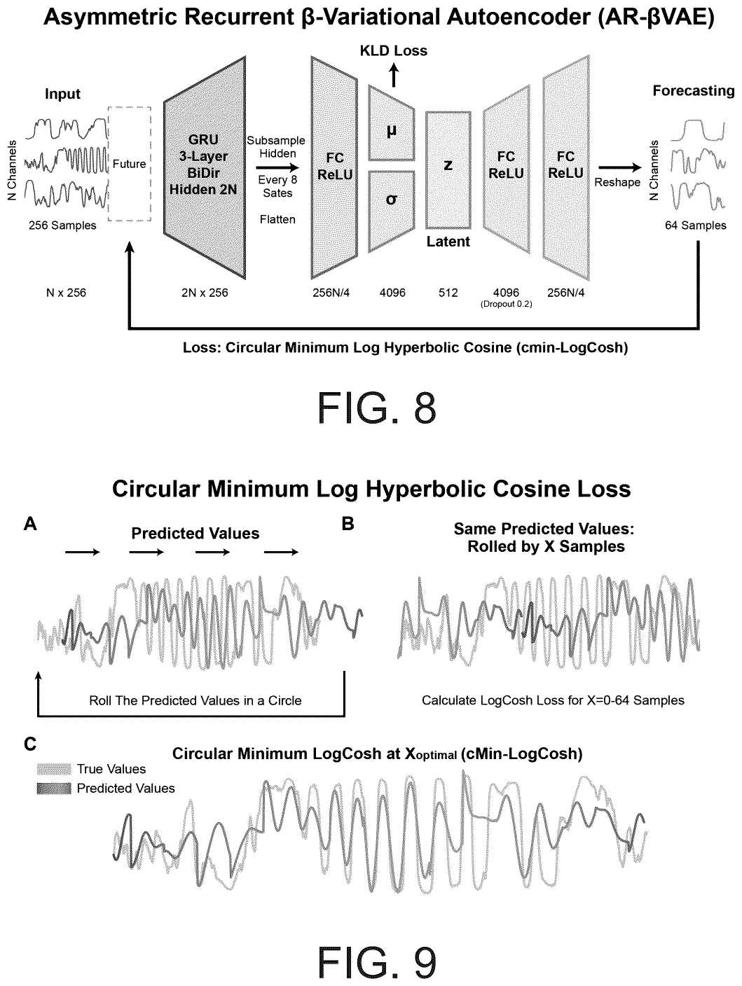

SEEG data was collected using Natus Neuroworks (Middleton, WI, USA) with a sampling rate of 512-2048 Hz. All data was resampled down to 512 Hz then filtered using MATLAB's (MathWorks inc., Natick, MA, USA) ‘filtfilt’ function to implement a zero-phase shift 5^{th} order Butterworth filter with passbands of 1 Hz-59 Hz, 61 Hz-119 Hz, and 121 Hz-179 Hz. An adjacent bipolar montage was applied across all SEEG leads and data was saved as 32-bit floating point precision Python pickle files (FIG. 7, element A).

### Zero-Centered One-Dimensional Histogram Equalization (ZHE).

Intracranial electrophysiological timeseries are often low amplitude with rare high amplitude signatures. This can make it difficult for a model to learn nuanced long-range characteristics because the histogram of the data distribution can have long tails (FIG. 7, element B, top panel) representing rare, but physiologically meaningful high voltage signatures. To provide more evenly distributed data suitable for machine learning models, a custom Zero-centered one-dimensional Histogram Equalization (ZHE) scheme was implemented (FIG. 7, element B). This scheme was motivated by traditional histogram equalization for images prior to model training. First, the signal is split into the positive and negative domains, then a 10,000-bin histogram is filled for each domain from 0 to the absolute value of highest voltage. Then the linear transfer function from the raw signal to the equalized signal is calculated using the cumulative distribution function for the positive and negative domain separately. The result is a signal that preserves the physiological meaning of zero, but has data evenly distributed between −1 and 1 (FIG. 7, element C). A prospective normalization scheme must be applied to not violate causality while validating the model on subsequent data epochs, thus all subject's timeseries were normalized to the first 24 hours of data. The histograms for subsequent data epochs will, as expected, not be exactly uniform (e.g. FIG. 7, element B, bottom panel).

### Brain-State Embedding Model.

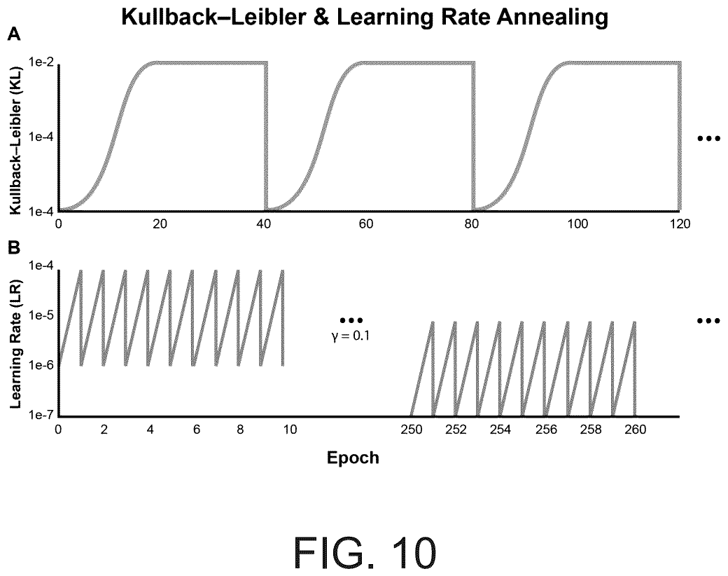

The ZHE data is then used to elucidate brain-states by compressing short (0.5 seconds) data epochs (FIG. 7, element D) into a 512-dimensional latent space (FIG. 7, element E) that is then used to forecast the immediate future (0.125 seconds) of all channel data simultaneously (FIG. 7, element F). This embedding is conducted using a custom Asymmetric Recurrent B-Variational Autoencoder (AR-BVAE), implemented in PyTorch, and trained on the first 70% of a subject's data (FIG. 8). A model was trained for each subject separately to accommodate unique SEEG implants and presumed unique neurophysiological brain-states experienced by each individual.

The AR-βVAE was architected to accept an N-channel (for this dataset, N ranged from 88-168 channels) by 256 sample (i.e. at 512 Hz sampling rate, 0.5 seconds of multichannel data). The top layer of the model utilizes a Gated Recurrent Unit (GRU) to interface directly with the input data and learn short- and long-range signal features. The GRU is three layers and bidirectional, with a resulting hidden dimension of 2N. The hidden state is then subsampled every eight states. In theory, this helps the GRU to maintain learning capacity and not be forced to forget data motifs across the entire 256 forward and 256 reversed sequence lengths. The subsampled hidden states are then flattened and fed into a β-VAE consisting of a fully-connected layer feeding into mean (μ) and log-variance (σ) layers followed by the standard noise-injection reparameterization trick to get ‘z’, the latent space. The latent space was regularized during training with Kullback-Leibler (KL) Divergence. The latent space of the variational autoencoder is set to 512 dimensions. The decoder outputs an N×64 sample forecast on all channels simultaneously. Thus, all N×256 input timepoints are compressed into 1×512 latent dimensions that best embed the necessary information to forecast the next Nx64 timepoints in the original input signal. A dropout ratio of 0.2 was used on the middle fully connected layer of the decoder to promote concise and meaningful latent embeddings.

### Circular Minimum Log Hyperbolic Cosine Loss.

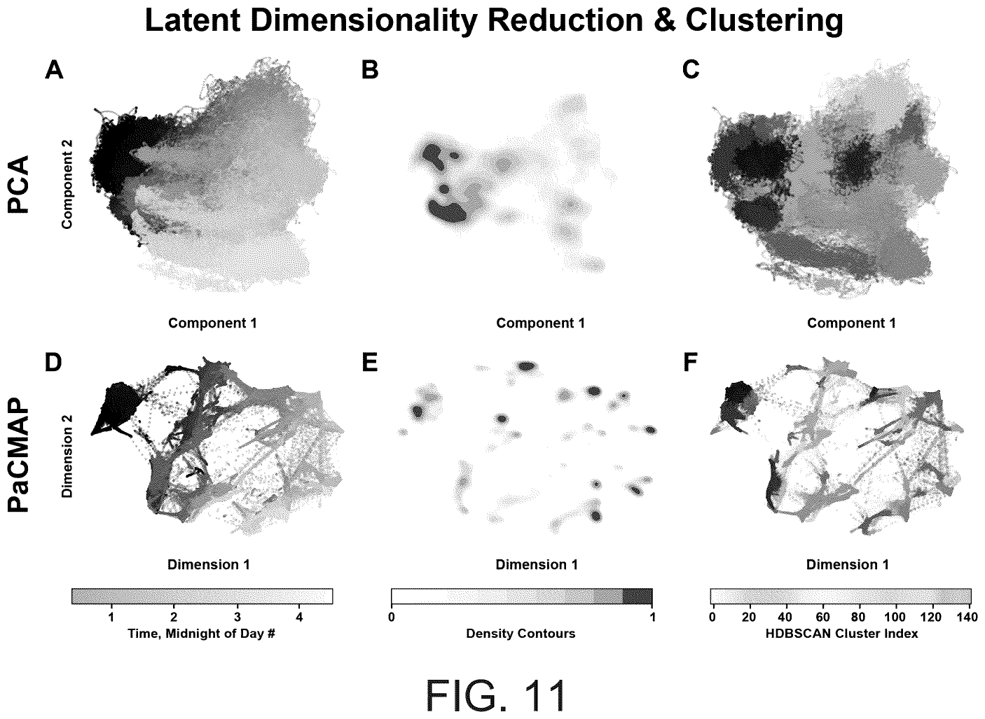

Of significance, the decoding portion of the β-VAE is asymmetric because the input GRU layer in the encoder cannot be easily reversed for the decoder. This creates an undesirable situation where slightly shifted input signals could require dramatically different embeddings due to the rigidity of the output fully-connected decoding layers—e.g., small time shifts in the input data could require very different outputs from the final fully connected layers. To accommodate this asymmetric intractability, a custom loss function (FIG. 9) was developed that allows for a varying time shift of the forecasted signals, termed Circular Minimum Hyperbolic Cosine Loss (cMin-LogCosh). This loss function was based on the hyperbolic cosine loss function as implemented in PyTorch by ‘auraloss’ package. Specifically, the N×64 predicted values are wrapped in a circle using ‘torch.roll’ in PyTorch with a stride of one sample (FIG. 9, elements A and B) and the LogCosh loss calculated every stride resulting in 64 individual loss values. The minimum LogCosh loss is deemed the cMin-LogCosh (FIG. 9, element C) and returned for backpropagation through the model.

### Training Considerations & Kullback-Leibler/Learning Rate Annealing.

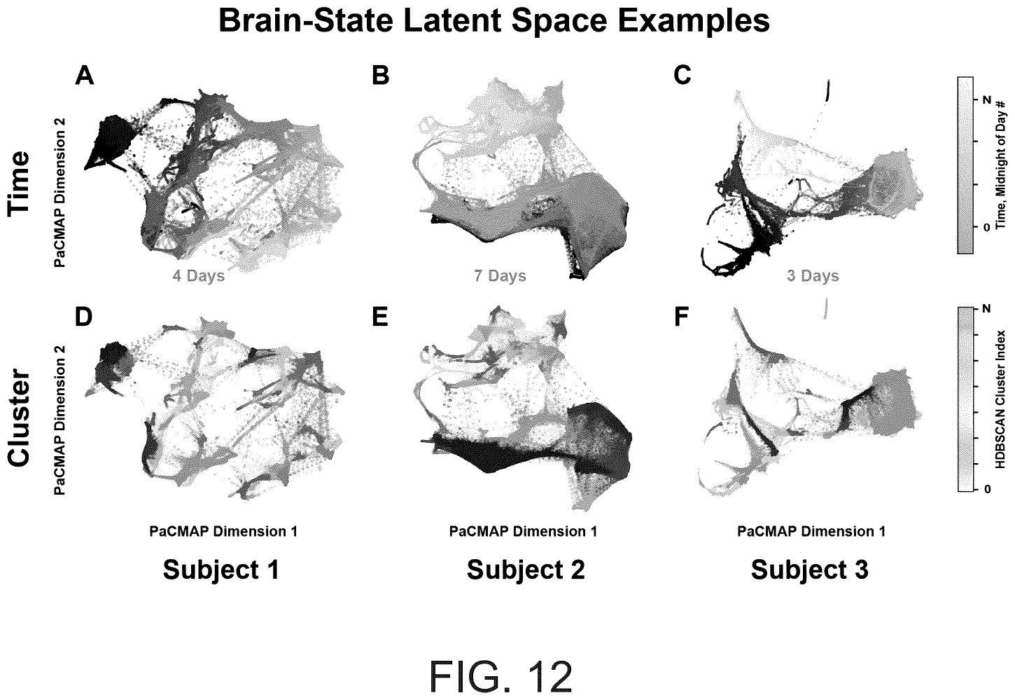

Each subject's model was trained by feeding in random N×256 epochs of data with a batch size of 64. A total of 640,000 random epochs and the models were each run to a total of 500 epochs, resulting in a total of 320M random epochs used for training. The first 70% of the patient's data was used for training and the remaining 30% was completely left out and used in the validation sections as described below. In an attempt to maximize biologically meaningful features extracted from the dataset, a sigmoidal KL annealing schedule was implemented with a 40 epoch period (FIG. 10, element A). Additionally, to increase model training stability, a learning rate (LR) annealing with a single epoch saw-tooth waveform and a gamma of 0.1 at 250 of the total 500 training epochs was implemented (FIG. 10, element B).

### Brain-State Interpretation: Dimensionality Reduction & Clustering.

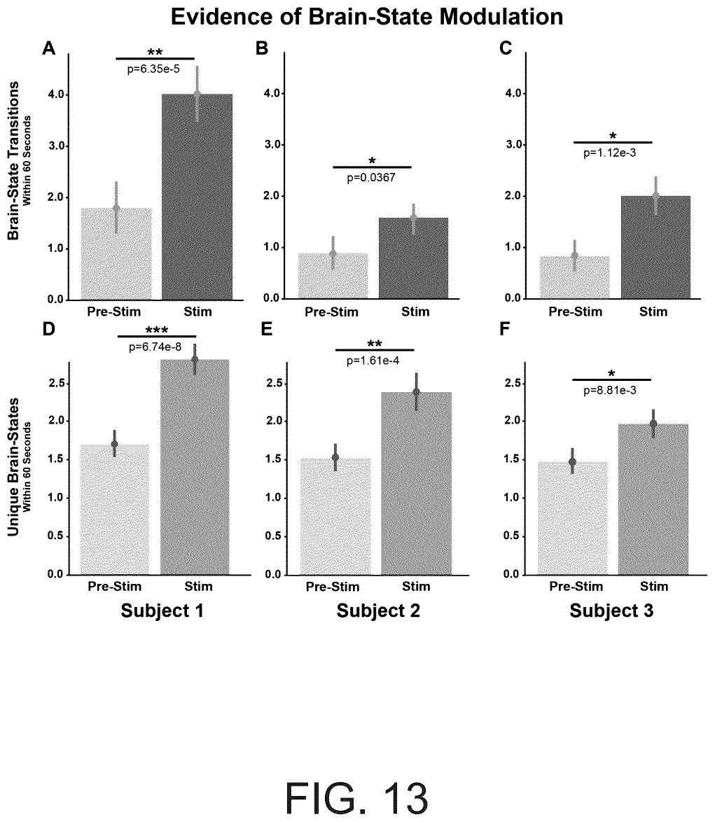

After model training has completed, the entire training and validation datasets (70/30%) are sequentially run through the model to get a continuous 512 dimensional latent-space representation of the data. These high-dimensional timeseries data are impossible for a human to interpret; thus, two steps are required before biologically meaningful analyses can be conducted: 1) Dimensionality reduction, 2) Brain-state clustering. To begin, dimensionality reduction is critical to interpreting the latent space architecture, but traditional methods like Principal Components Analysis (PCA) fail to separate brain-states into any discernible structure (FIG. 11, elements A-B). Thus Pairwise-Controlled Manifold Approximation and Projection (PaCMAP), a dimensionality reduction algorithm and successor to the popular Uniform Manifold Approximation and Projection (UMAP) were used, that has demonstrated enhanced ability to capture global data structure, which is critical for analyzing large-timescale brain states (FIG. 11, element D). Before feeding the latent space data into PaCMAP, the latent space data were smoothed the 512-dimensional latent data with a 10-second averaging window, and 1 second stride—it was found that this greatly increases the signal-to-noise ratio for the manifold approximation. The PaCMAP hyperparameters that differed from default were as follows: Distance: cosine, MN_ratio 2.0, FP_ratio 0.5 to enhance global structure definition of the reduced space.

Next, differing densities of data appeared to be present in the data, as can be appreciated in the iso-density contours in FIG. 11, elements B and E. Thus, the PaCMAP reduced dimensional space was clustered using Hierarchical Density-Based Spatial Clustering of Applications with Noise (HDBSCAN). Of note, two separate runs of PaCMAP were conducted, the first is the 512 dimensional latent space reduced to a ten-dimensional space that was fed into HDBSCAN, and the second is a two-dimensional space for direct visualization in the plots shown. This was done to allow HDBSCAN to have access to an enriched ten-dimensional feature set that could help distinguished unique brain states without being limited to the simplified two-dimensional space. The hyperparameters that differed from default for HDBSCAN were as follows: min_cluster_size 200 (each data point is a 10 second average of latent data), min_samples 100. After dimensionality reduction and clustering, distinct brain-state groupings within the latent space become apparent in the 2D PaCMAP visualization (FIG. 11, element F), that would have otherwise been indistinguishable by PCA (FIG. 11, element C). Of note, the PaCMAP and HDBSCAN models were built with only training data.

Next, using the trained AR-βVAE, PaCMAP and HDBSCAN models, the completely withheld validation data can be run through the pipeline to observe the most-likely brain-state cluster for data the models have never previously seen. This flow of data through the pretrained models is how a real-time feedback signal would be generated for a closed-loop neuromodulation device. An ‘adaptive’ paradigm would arise from continuously updating these models based on new data to refine the brain-state estimation in real time.

### Brain-State Model Validation: Perturbability & Biological Relevance.

The generation of clustered latent spaces from epiphenomenal physiological timeseries has little meaning to potential closed-loop neuromodulation applications without two important forms of validation: 1) Evidence of brain-state perturbability with neurostimulation, 2) Evidence of biological relevance of brain-states. Both of these validation conditions must be reasonably met to proceed with a closed-loop neuromodulation paradigm. However, the exact criteria that dictates adequate validation is nebulous-especially establishing biological relevance because this is subject and disease specific. For example, the latent space of a person with epilepsy may differ greatly from a person with major depressive disorder. Or more nuanced, the difference between two people with epilepsy who experience different seizure types, take different medications, and are different ages. Thus, the following quantitative and qualitative techniques were implemented to capture the subject-to-subject variability in brain-state organization.

Brain-State Perturbability with Low-Frequency Neurostimulation.

To capture possible effects of neurostimulation, previously collected single-pulse electrical stimulation (SPES) paradigm conducted with 71 of 118 pre-surgical epilepsy patients were utilized. This stimulation paradigm was not designed to maximize any possible brain-state modulation and consists of only 1 Hz, 300 microsecond biphasic pulses in trains of 10 seconds and at current levels of 1-5 milliamps. Thus, these SPES sessions offer a conservative insight into the effects of a low-energy stimulation paradigm's effects on brain-state. The SPES data epoch (approximately 1-2 hours per subject) was excluded from training. Inference of the SPES epoch was conducted without manual stimulation artifact removal to minimize potential bias and simulate a real time device implementation-artifact is greatly mitigated by the ZHE normalization scheme and with the brevity of SPES artifact accounting for approximately 0.1%-0.2% of any smoothed 10 second window. For continuous stimulation paradigms with a higher duty cycle, this technique may need to be amended.

To quantify any effects of neurostimulation, two high-level metrics: 1) number of brain-state transitions within a 60 second window, 2) number of unique brain-states occupied within a 60 second window were implemented. These metrics serve as a baseline validation to capture coarse information about the effects of neurostimulation. Importantly, due to the architecture of each model in a subject-specific manner, it is ill-posed to compare these metrics across subjects. Thus, a bootstrapping technique was implemented to estimate the statistical significance for each subject. Specifically, the ‘state-transitions’ and ‘unique-clusters’ metrics were evaluated for all non-overlapping 60-second windows within the SPES epoch, then calculated the metrics for an equivalent number of random 60-second epochs in the pre-SPES epoch. From these bootstrapped runs, 95% confidence intervals and two-sample t-tests can be conducted with Bonferroni-Holm multiple comparison correction.

### Biological Relevance of Brain-States.

Thus far, all methodology has been left agnostic to any specific neurological disease. However, for the purpose of determining the biological relevance of the elucidated brain-states, the latent spaces must be interpreted in the specific context of the cohort's neurophysiological pathology-drug-resistant epilepsy. This type of validation relies on qualification of the latent space structure with detailed clinical information known about each subject. Most importantly for this cohort, the exact timing and type of all electroclinical seizures. With these events as ground truth, it can be worked backwards to overlay meaning to the brain-states that precede and follow these events.

With the epilepsy brain-state postulates in mind from Table 1, three qualitative questions were asked on the training data and validation data withheld from each subject's trained model: 1) Does pre-ictal, ictal, and post-ictal activity in training data aggregate together in the latent space in a meaningful structure, and are any patterns based on different seizure types present? 2) Does completely withheld validation data for similar electroclinical epileptic events map to the expected latent space regions based on training data latent space assessment? 3) Can seizure propensity be assigned to the HDBSCAN clusters and plotted on a timeline for training data which can then be mapped to validation data?

To address the first and second validation questions, the entire training and validation datasets were run through the trained AR-βVAE model and the latent spaces for each subject were labeled with known ictal events. The latent space density iso-contours were concurrently plotted to assess the most common brain-states during the training and validation epochs. Next, the latent architectures during ictal and interictal periods were analyzed for meaningful aggregation of known electroclinical states. Finally, to assess the seizure propensity of the latent space clusters (i.e. brain-states), a timeline of cluster assignments for the training dataset was plotted against known ictal events. An exponentially decaying seizure propensity scoring mask (see Eq. 1, where ‘ť’=sample count to seizure at sampling rate of 512 Hz, and d=decay factor of 20,000) was assigned to the timeline for all ictal events, with the maximum mask value at each timepoint was used to account for temporally adjacent ictal events.

Seizure Propensity Scoring Mask=e^{(-t/d)}( Eq 1.

The brain-state clusters were then ranked by the mean scoring mask value of all time periods during which the cluster was assigned. With the cluster seizure propensity score assigned from the training dataset, the validation scored timeline can be plotted to assess the ability of the model to discern potentially high seizure risk brain-states on data the model has never seen before.

### Results

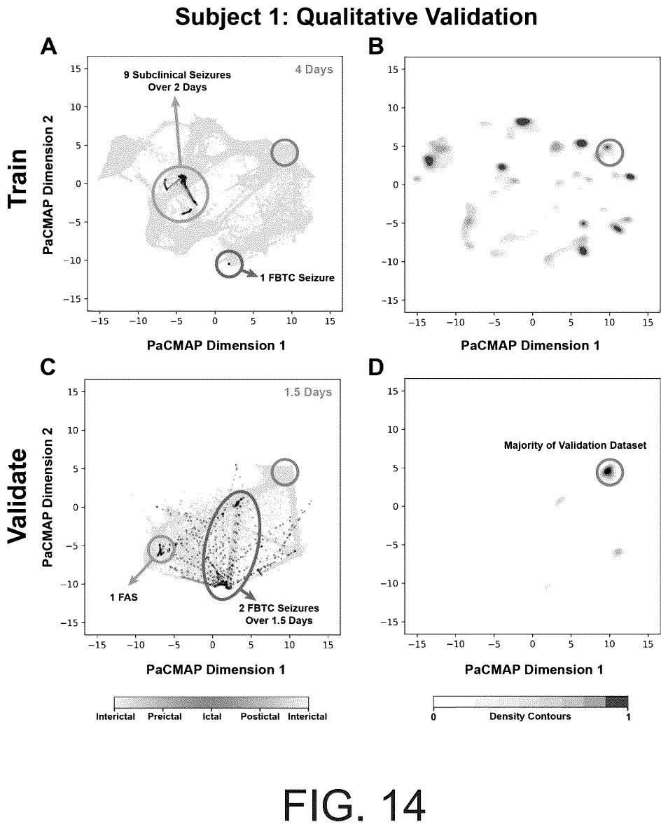

The main goal of this experiment is to provide an initial validation of the methods of brain-state modeling using high-level quantitative and qualitative metrics to assess proof-of-concept suitability for an adaptive closed-loop neuromodulation paradigm. Utilizing this modeling process, example latent spaces and brain-state clusters can be seen in FIG. 12.

### Brain-State Observed to be Significantly Perturbed by Low-Frequency Stimulation.

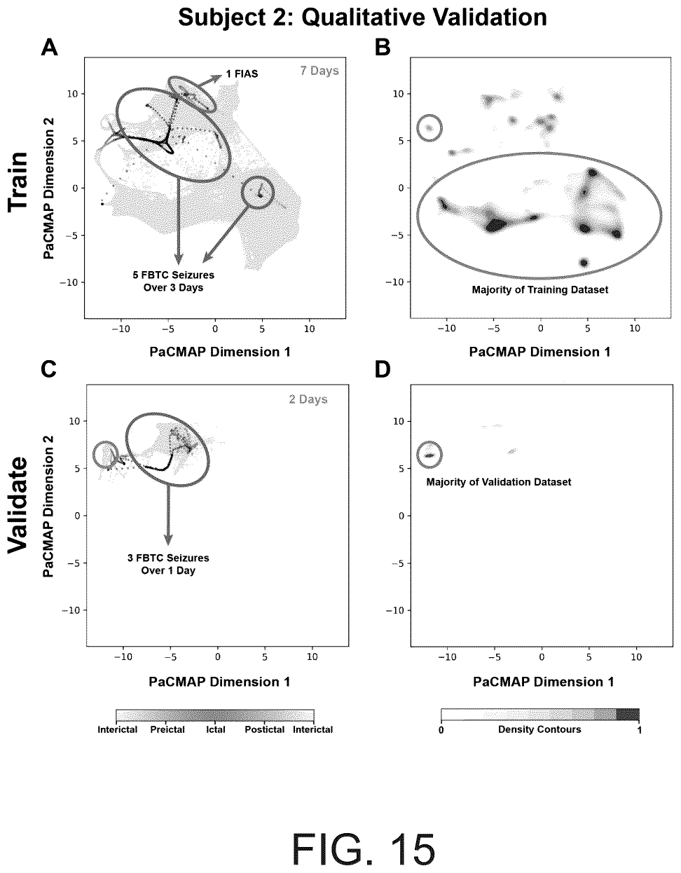

The results of the neurostimulation perturbation analysis for these example subjects can be seen in FIG. 13. SPES displayed evidence of significant brain-state perturbation, with the mean number of brain-state transitions observed to be 177%-250% higher during the SPES epoch: Bootstrapping t-test p-values ranged from 6.35e-5 to 0.0367 for the average number of brain-state transitions during a given 60 second window in the pre-SPES vs. SPES epochs. Furthermore, the increase in brain-state transitions during the SPES epochs were not simply observed to be oscillations between the same brain-states because the number of unique brain-states increased by 127%-163% during the SPES epochs, with p-values ranging from 6.74e-8 to 8.81e-3. These results indicate that even though SPES is a low-energy stimulation paradigm, it may still be significantly altering the brain-state as captured by the proposed brain-state modeling process.

### Brain-States Appear to be Biologically Relevant Based on Inspection of Latent Space Architecture.

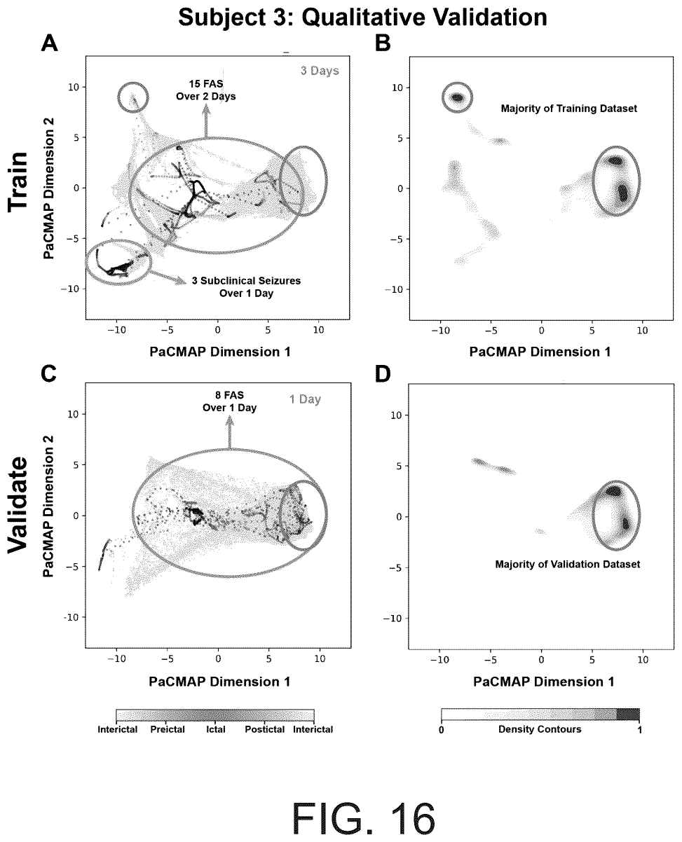

Beyond the ability to capture possible effects of neuromodulation, the brain-state modeling process must capture biologically meaningful information for a given neuropathology to be of clinical utility. To assess biological relevance, the qualitative assessment of the latent spaces for three diverse subjects with epilepsy was outlined (FIGS. 14-16). Two subjects experienced focal to bilateral tonic-clonic (FBTC) seizures during SEEG recording (Subjects 1-2) and the third (Subject 3) experienced only subclinical and focal aware seizures (FAS). Of further interest, Subjects 1-2 had multiple seizure-free days prior to the first seizure captured on SEEG recording, whereas Subject 3 had multiple subclinical and FAS immediately on the first day of recording. These are important factors to consider when looking at the latent spaces for these subjects.

To begin, Subject 1's latent space for the training data (FIG. 14, elements A-B) displays a distinct grouping of nine subclinical seizures that occurred over a two-day period. Furthermore, the sole FBTC seizure present in the training dataset is well separated from this subclinical aggregation, providing evidence that the pre-ictal brain-state prodrome for the FBTC seizure differs to that of the subclinical seizures. However, these observations are for the data that the model had seen during training, thus analysis of the validation data completely withheld from the model training is of more interest. The validation latent spaces for Subject 1 (FIG. 14, element C) at first appear noisy, but when looking at the density iso-contours of brain-states (FIG. 14, element D), the majority of the validation brain-states were distant to areas of the latent space that were labeled as ictal in the training latent space. Furthermore, the two FBTC seizures present in the validation data mapped well to the training FBTC latent space despite only a single FBTC seizure being present for training. Finally, a new seizure type was present in the validation data (FAS) that mapped to a distinct location in the latent space near the subclinical aggregation despite this seizure type never having been seen by the model for training. This subject serves as a nice example of the potential variety in pre-ictal brain-states for different types of seizures, as outlined in the implications of Postulate 1.

The next example subject also had many seizure-free days prior to the first electroclinical event, however this patient's training data had five FBTC seizures present in the training data (i.e., first 70% of data), thus this subject's model was exposed to many more brain-state evolutions toward a FBTC seizure (FIG. 15, elements A-B). This is likely reflected in the tight aggregation of FBTC seizures in the validation data (FIG. 15, element C) to the expected highest density of FBTC seizures in the training data (FIG. 15, element A). Notably, the FTBC seizure in the training latent space that is most distant from the other four FBTC seizures was the first electroclinical event that the patient experienced during recording-perhaps triggering the subject to enter the high seizure propensity brain-states that resulted in four FBTC seizures over the next 2 days. An observation that provides further evidence for the subject's overall brain-state shift throughout recording is the very different latent space occupancy during training (FIG. 15, element B) compared to validation (FIG. 15, element D). This serves as a good example of potentially different pre-ictal brain-state evolutions that can occur, as outlined in Postulate 2. Finally, this patient also experienced a focal impaired-aware seizure (FIAS) that mapped adjacent to the FBTC seizures in the training latent space, but no FIAS were present in the validation data to assess generalizability.

The last example, Subject 3, had a significantly different electroclinical timeline of ictal events during SEEG recording, which is reflected in the training and validation latent spaces (FIG. 16). The first observation is that the iso-density contours for training (FIG. 16, element B) and validation (FIG. 16, element D) are similar—this indicates that unlike Subjects 2 & 3, this subject likely did not experience a significant shift in the constellation of brain-states during recording. Thus, this subject serves as an exemplary case for the implications of Postulate 3 that there may be no stable interictal brain-state mapped into the latent space if the subject is experiencing a high ictal burden during recording. As suspected, unlike Subjects 1 & 2 with a more concentrated aggregation of ictal activity in the latent space, Subject 3's latent space is dominated by numerous FAS and subclinical seizures (FIG. 16, element A). Importantly, the peri-ictal activity for the three subclinical and 15 FAS in the training data did map to different areas of the latent space, but the FAS tend to dominate the majority of the space. Evidence for this patient-specific hypothesis is observed in the validation latent space (FIG. 16, element D), where the eight FAS present in the validation data occupy a similar, but also expansive, area of the latent space similar to the training data. For the purposes of brain-state modeling, this patient would have benefitted from a longer recording time to hopefully capture periods of lower seizure burden. Thus, as Postulate 3 states, it may be difficult to extrapolate a ‘stable’ interictal brain-state that is far in latent space from ictal activity if no stable state has been previously observed.

### Seizure Propensity Timeline Demonstrates Large-Timescale Transition to High Ictal Burden.

An important feature of a potential closed-loop neuromodulation device for drug-resistant epilepsy is the ability to sense large-timescale shifts in brain-state. Without this ability, the device operates in one of two paradigms: 1) The “too little too late” paradigm where only an immediate pre-ictal signature can be detected and stim can be delivered, or 2) The device inappropriately characterizes large-timescale brain-states and thus operates in essentially an open-loop fashion with inadequate feedback signaling.

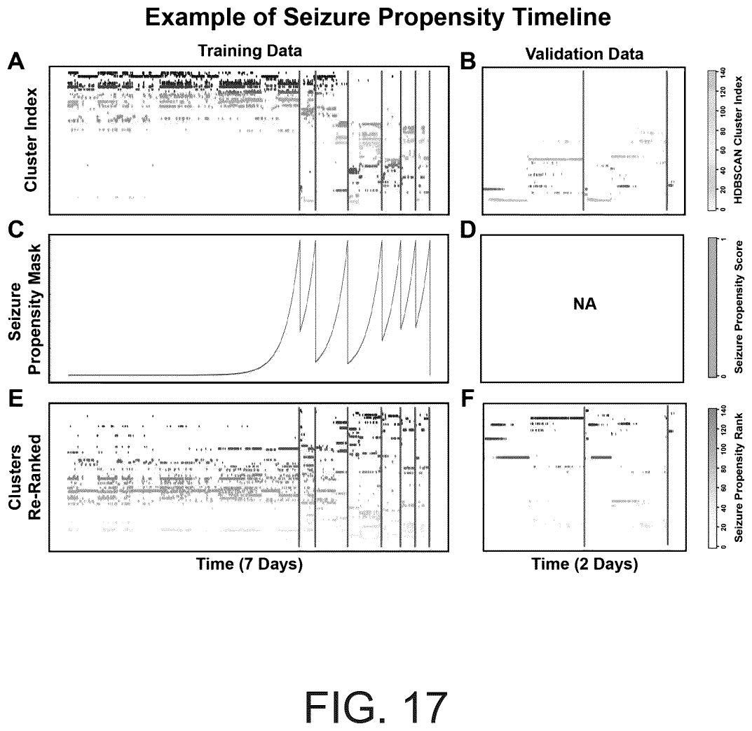

An example of the proposed brain-state modeling process ability to capture large-timescale brain-states is shown in FIG. 17 where example Subject 2's seizure propensity timeline is plotted for training and validation data. This subject serves as a good example because the training data consists of many days of seizure-free recordings preceding multiple days of frequent FBTC seizures (FIG. 17, element A), which were continued in the validation data (FIG. 17, element B). Thus, using the seizure propensity masking technique (FIG. 17, element C), the brain-state clusters can be ranked to align with estimated seizure-propensity within an arbitrary timeframe (FIG. 12, element E), high values indicate high risk for a seizure. The validation timeline for this subject appropriately suggests that this patient spent most of the two days of validation recordings in a high seizure propensity state-which is in alignment with the two FBTC seizures this patient experience during validation. It is important to note that these are not immediately pre-ictal states and could be operated on by a neuromodulation device on the order of hours-days in an attempt to neuromodulate this subject through their mapped latent space to previously detected clusters with a lower seizure propensity.

From the above description, those skilled in the art will perceive improvements, changes, and modifications. Such improvements, changes and modifications are within the skill of one in the art and are intended to be covered by the appended claims.

The following is claimed:

1. A system for closed-loop neuromodulation to treat a condition of a brain, the system comprising:
at least one recording electrode configured to record conduction data from at least a portion of the brain;
at least one stimulating electrode configured to apply an electrical signal, generated and configured by a generator, to at least another portion of the brain, wherein the electrical signal comprises at least one parameter; and
a controller in electrical communication with the at least one recording electrode and the generator, the controller comprising a non-transitory memory configured to store instructions and a trained patient-specific model and a processor configured to execute the instructions and the trained patient-specific model to:
receive the conduction data at a time;
project the conduction data through the trained patient-specific model to determine a brain state at the time;
update the at least one parameter of the electrical signal based on a propensity of the brain state at the time to cause an effect of the condition of the brain; and
update the trained patient-specific model to include the brain state at the time and an effect of the updated the at least one parameter of the electrical signal on the brain state at the time.

2. The system of claim 1, wherein the processor is configured to process the conduction data at the time into brain state data at the time, wherein the brain state data is compatible with the trained patient-specific model before being projected through the trained patient-specific model to determine the brain state at the time.

3. The system of claim 2, wherein the conduction data at the time is filtered into multi-dimensional time series data, equalized using a zero-centered one-dimensional histogram equalization to form equalized time series data, and then embedded into a multi-dimensional latent space using an asymmetric recurrent variational autoencoder to form the brain state data at the time.

4. The system of claim 3, wherein a dimensionality of the brain state data at the time is reduced and the reduced dimensionality brain state data at the time is clustered before one or more brain state groupings are identified that the brain state data corresponds to in the trained patient-specific model.

5. The system of claim 4, wherein the brain state data at the time is reduced with a pairwise controlled manifold approximation and projection (PaCMAP) into at least one lower dimension of data and then clustered with a Hierarchical Density Based Spatial Clustering of Applications of Noise (HDBSCAN).

6. The system of claim 1, wherein the processor is configured to determine the propensity of the brain state at the time to cause the effect of the condition of the brain based on brain state data at the time being projected through the trained patient-specific model, wherein the trained patient-specific model is trained over brain state data from a previous time period of at least one hour.

7. The system of claim 6, wherein the propensity is determined by comparing the brain state data at the time to identified distinct brain state groupings with known outcomes in the trained patient-specific model.

8. The system of claim 1, wherein the processor executes the instructions to train the trained patient-specific model using previous conduction data of the patient over a previous time period of at least one hour to form a multi-dimensional latent space with identified brain state groupings having known outcomes.

9. The system of claim 8, wherein the previous conduction data of the patient over the time period comprises at least one channel of time series data, wherein the at least one channel corresponds to the at least one recording electrode.

10. The system of claim 1, wherein the brain state at the time corresponds to a propensity for a future seizure.

11. The system of claim 10, wherein the propensity of the brain state at the time corresponds to a likelihood to cause a seizure on a day in the future if the electrical signal is not modulated.

12. The system of claim 1, wherein the electrical signal provides a low-energy stimulation to the other portion of the brain.

13. A method for closed-loop neuromodulation to treat a condition of a brain, the method comprising:
receiving, by a system comprising a processor, conduction data at a time from at least one recording electrode in communication with the processor, wherein the at least one recording electrode records conduction data from at least a portion of the brain;
projecting, by the system, the conduction data at the time through a trained patient-specific model to determine a brain state at the time;
updating, by the system, at least one parameter of an electrical signal based on a propensity of the brain state at the time to cause an effect of the condition of the brain, wherein the processor is further in communication with at least a generator that generates the electrical signal and provides the electrical signal to at least one stimulation electrode that applies the electrical signal to at least another portion of the brain; and
updating, by the system, the trained patient-specific model to include the brain state at the time and an effect of the application of the updated the at least one parameter of the electrical signal on the brain state at the time.

14. The method of claim 13, further comprising determining, by the system, the propensity of the brain state at the time to cause the effect of the condition of the brain based on brain state data at the time being projected through the trained patient-specific model, wherein the trained patient-specific model is trained over brain state data from a previous time period of at least one hour.

15. The method of claim 13, wherein the condition of the brain is a neurological pathology.

16. The method of claim 15, wherein the neurological pathology is epilepsy.

17. The method of claim 13, further comprising processing the conduction data at the time into brain state data at the time, wherein the brain state data is compatible with the trained patient-specific model before being projected through the trained patient-specific model to determine the brain state at the time.

18. The method of claim 13, wherein the conduction data at the time is filtered into multi-dimensional time series data, equalized using a zero-centered one-dimensional histogram equalization to form equalized time series data, and then embedded into a multi-dimensional latent space using an asymmetric recurrent variational autoencoder to form the brain state data at the time.

19. The method of claim 18, further comprising reducing a dimensionality of the brain state data at the time and clustering the reduced dimensionality brain state data at the time before one or more brain state groupings are identified that the brain state data corresponds to in the trained patient-specific model.

20. The method of claim 19, further comprising reducing the brain state data at the time with a pairwise controlled manifold approximation and projection (PaCMAP) into at least one lower dimension of data and then clustered with a Hierarchical Density Based Spatial Clustering of Applications of Noise (HDBSCAN).
